(19) United States
(12) Patent Application Publication
(10) Pub. No.: US 2006/0134096 A1
(43) Pub. Date: Jun. 22, 2006

(54) Compositions and methods for medical use of graphene-containing compositions

(75) Inventor: Viktor I. Petrik, S. Petersburg (RU)

Correspondence Address: ROBERT D. FISH;RUTAN & TUCKER LLP, 611 ANTON BLVD 14TH FLOOR, COSTA MESA, CA, 92626-1931, US

(73) Assignee: SupraCarbonic, LLC

(21) Appl. No.: 11/021,870

(22) Filed: Dec. 22, 2004

(51) Int. Cl. A61K 33/44

(52) U.S. Cl. 424/125; 977/906

(57) ABSTRACT

Non-porous carbon materials that are materials other than a fullerene or a nanotube are employed for medical use, wherein the carbon material has a smallest dimension of less than 100 nanometer. In preferred aspects, the material is topically used on a wounds, orally administered as sorbent for various toxins, or employed as a sorbent in hemodialysis.

### FIELD OF THE INVENTION

The field of the invention is carbon nanostructures, carbon nanostructure-containing materials, and their manufacture.

### BACKGROUND OF THE INVENTION

Medical use of activated charcoal has been reported for several centuries, and depending on the use, various forms of activated charcoal are available. For example, powdered activated charcoal is frequently orally used where a person has ingested a toxic compound. Such use is typically inexpensive and relatively effective. However, oral administration of activated charcoal also removes compounds other than the toxic compounds and long-term administration is often not advised. Alternatively, fullerene-containing preparations can be used as adsorbents for certain toxins. However, as fullerenes are only marginally removed from the circulatory system, repeated use is often problematic. For example, long-term administration was reported to cause significant nephrotoxicity.

Activated charcoal is also known as in topical treatments, and especially for purulent and exuding wounds. For example, optionally silver impregnated carbonized and activated knitted viscose rayon fabric (sold as Actisorb, or Actisorb Plus) is used in a nylon sleeve for wound treatment. In other examples, an activated charcoal cloth and an absorbing layer of mixed fibers is used in combination with a wound contact layer of alginate and carboxymethylcellulose fibers (sold as CarboFlex) to absorb exudates and/or reduce wound odor. In other known topical treatments, a non-woven fabric is impregnated with activated carbon granules, and is enclosed in a polyurethane foam for wound contact (sold as Lyofoam C).

While such known activated charcoal compositions have various beneficial properties and are often relatively inexpensive to manufacture, several difficulties nevertheless remain. Among other things, due to the porous nature of the activated charcoal, selectivity of the charcoal for the toxins is typically low. Moreover, and depending on the pore size, activated charcoal can serve as a growth substrate for wound-associated microorganisms.

Therefore, while there are numerous materials and methods for medical use of carbon-based materials known in the art, all or almost all of them suffer from one or more disadvantages. Consequently, there is still a need to provide improved compositions and methods for medical use of carbon-based materials.

### SUMMARY OF THE INVENTION

The present invention is directed to compositions and methods in which a non-porous carbon other than a fullerene or a nanotube is used for medical use. Typically, the carbon has a smallest dimension of less than 100 nanometer, and is topically, orally, and/or extracorporally administered. Preferably, contemplated the non-porous carbon has a smallest dimension of less than 20 nanometer, and most preferably comprises graphene, which is typically present in contemplated compositions at an amount of at least 10 wt %, and most typically at least 50 wt %.

In one aspect of the inventive subject matter, the composition is formulated for topical administration. Thus, suitable compositions may further comprise an information that informs a person to apply the composition to an affected area (e.g., open wound, which may be infected, purulent, and/or exuding, a burned area, which may be infected, purulent, and/or exuding, or an area with an allergic reaction). Alternatively, the composition may be formulated for oral administration, and can therefore further include an information that informs a person to administer the composition to someone suffering from an intoxication, diarrhea, and/or food poisoning. In still further contemplated uses, compositions according to the inventive subject matter may also be employed for dialysis or similar processes where blood, serum, or other body fluids contact the composition outside a person's body. In such uses, an information is typically associated with the composition that information informs a person that the composition reduces the concentration of uric acid, lactic acid, and/or creatinin in blood or serum.

In another aspect of the inventive subject matter, a wound dressing includes contemplated compositions, which are typically at least partially enclosed in a carrier, wherein at least one layer of material (e.g., gauze, alginate, and/or synthetic polymer) is disposed between the wound and the composition.

In yet another aspect of the inventive subject matter, a method of treating a medical condition in a subject includes one step in which contemplated compositions are administered to a person in need thereof in an amount effective to improve at least one symptom of the condition. Most typically, contemplated conditions include wounds and burns (which may be infected, purulent, and/or exuding), topical allergic foci (e.g., bee sting, etc.), intoxications, diarrhea, food poisoning, renal dysfunction, hepatic dysfunction, hyperuricemia, lactic acidosis, and/or hypercreatininemia.

Various objects, features, aspects and advantages of the present invention will become more apparent from the figures and the following detailed description of preferred embodiments of the invention.

### BRIEF DESCRIPTION OF THE DRAWING

FIG. 1A is an exemplary electronmicrograph depicting graphene produced using methods according to the inventive subject matter.

FIG. 1B is a detail view of the graphene of FIG. 1B at a higher magnification.

FIG. 2 is an exemplary schematic wound dressing according to the inventive subject matter.

### DETAILED DESCRIPTION

The inventors surprisingly discovered that non-porous nanostructured carbon and other carbonaceous materials can be effectively employed in the treatment of numerous internal and/or external conditions.

In especially preferred aspects, contemplated medical compositions include a non-porous carbon (having a structure other than a fullerene or a nanotube), in which the non-porous carbon has a smallest dimension of less than 100 nanometer (nm), more typically of less than 50 nm, even more typically of less than 20 nm. The smallest dimension is typically the thickness (or average thickness) of the non-porous carbon. Most typically, the non-porous carbon comprises graphene, which may be present in contemplated compositions at any desired amount. It is generally preferred, however, that the graphene is present in an amount of at least 1 wt %, more preferably in an amount of at least 10 wt %, even more preferably in an amount of at least 50 wt %, and most preferably in an amount of at least 80 wt %. The term “medical composition” as used herein refers to a composition of matter that is exclusively used in a treatment of a condition of a mammal, and especially a human. Therefore, a composition of matter that is used in electron emission, hydrocarbon adsorption, or other non-medical uses will not fall within the scope of the definition provided herein.

As also used herein, the term “non-porous carbon” refers to a carbonaceous material (i.e., a material that comprises at least 80 atom % carbon) having a porosity (i.e., void space within the material itself) of less than 5 vol %, and even more typically of less than 2 vol %. For example, a material having a total volume of 10 cubic micrometer is considered non-porous is that material has a total pore volume of less than 0.5 cubic micrometer. It should be noted that the annular space defined by a carbocyclic ring is not considered a pore under the definition provided herein. Also, where a material has a contorted shape (e.g., a graphene layer in a wrinkled, sheet-like configuration) within a given volume, the void space between the material in that volume is not considered a pore under the definition provided herein.

As also used herein, the term “graphene” refers to a molecule in which a plurality of carbon atoms (e.g., in the form of five-membered rings, six-membered rings, and/or seven-membered rings) are covalently bound to each other to form a (typically sheet-like) polycyclic aromatic molecule. Consequently, and at least from one perspective, a graphene may be viewed as a single layer of carbon atoms that are covalently bound to each other (most typically sp² bonded). It should be noted that such sheets may have various configurations, and that the particular configuration will depend (among other things) on the amount and position of five-membered and/or seven-membered rings in the sheet. For example, an otherwise planar graphene sheet consisting of six-membered rings will warp into a cone shape if a five-membered ring is present the plane, or will warp into a saddle shape if a seven-membered ring is present in the sheet. Furthermore, and especially where the sheet-like graphene is relatively large, it should be recognized that the graphene may have the electron-microscopic appearance of a wrinkled sheet.

It should be further noted that under the scope of this definition, the term “graphene” also includes molecules in which several (e.g., two, three, four, five to ten, one to twenty, one to fifty, or one to hundred) single layers of carbon atoms (supra) are stacked on top of each other to a maximum thickness of less than 100 nanometers. Consequently, the term “graphene” as used herein refers to a single layer of aromatic polycyclic carbon as well as to a plurality of such layers having a thickness of less than 100 nanometers. Typically, the dangling bonds on the edge of the graphene are saturated with a hydrogen atom. The term “about” where used in conjunction with a numeral refers to a numeric range of ±10% of the numeral, inclusive. For example, the term “about 100” refers to a numerical value of between 90 and 110, inclusive.

As further used herein, the term “carbon nanotube” refers to a cylindrical single- or multi-walled structure in which the wall(s) is (are) predominantly composed of carbon, wherein the diameter may be uniform or decreasing over the length of the nanotube. In some instances, the carbon nanotube can be curved, and is therefore also termed “carbon nanohorn”.

Surprisingly, the inventors discovered that the non-porous surface of nanostructured materials, and especially materials with a smallest dimension of less than 100 nm effectively bind (typically in a non-covalent manner) numerous medically relevant compounds, fluids, and/or organisms from a variety of physiological environments. Among various other nanostructured materials, selected carbonaceous materials, and particularly graphene exhibited superior binding characteristics. Viewed from another perspective, non-porous surfaces of carbonaceous materials with generally flat configuration (i.e., materials in which the first and second dimensions are substantially larger [e.g., at least 1000-fold] than the third dimension) are particularly effective, and have in most cases a smallest dimension of less than 500 nm, and more typically of less than 300 nm, even more typically of less than 200 nm, and most typically of less than 100 nm.

While not wishing to be bound by any hypothesis or theory, the inventors contemplate that the remarkable adsorption characteristics of the carbon materials according to the inventive subject matter is at least in part due to the relatively large, hydrophobic (i.e., lipophilic) surface of the non-porous carbon. Moreover, graphene has been demonstrated to act as an electron donor as well as an electron acceptor, which may further explain the relatively high affinity of certain compounds to the materials. It should further be recognized that where the non-porous carbon is graphene, the binding characteristics may also be influenced by orbital strain of the graphene sheet where the sheet is in a configuration other than a flat configuration. Similarly, pi-stacking may add to the unusual binding effects.

Among other compounds, the inventors discovered that the compositions according to the inventive subject matter have a high affinity for linear and branched hydrocarbons, which may optionally include one or more functional groups (e.g., carboxyl, hydroxyl, amino, etc.). For example, physiologically relevant compounds that are effectively bound by the compositions presented herein include lactic acid, butyric acid, caproic acid, methanol, ethanol, butanol, cadaverin, spermidine, etc. Further compounds identified with high affinity to the non-porous carbon include uric acid and creatinine. Remarkably, however, when the non-porous materials were employed as a sorbent during hemodialysis, electrolytes, glucose, lipids (bilirubin and liver enzymes) remained unaffected. Therefore, it is contemplated that topical and/or systemic administration of contemplated compounds may provide beneficial effects where the bound compound contributed to a disease state or inhibits healing. Specific exemplary uses are provided below in the section entitled “Examples”.

Furthermore, the inventors observed a direct and indirect antimicrobial effect using the compositions according to the inventive subject matter. For example, bacterial counts in wounds exposed to the non-porous carbon were dramatically reduced without negative interference with wound healing as compared to standard antiseptic wound treatment. Such effect may be due to formation of an insulating layer that physically protects the wound surface, but also due to binding of microorganisms to the non-porous surface.

Based on these and other observations, the inventors contemplate compositions that will include in addition to the non-porous carbon various other components that may contribute to a desirable physiological effect, and/or provide other advantageous properties to the composition. For example, suitable other components include antibacterial agents (e.g., lysozyme, antibiotic or bacteriostatic agent, etc.), antiviral agents (e.g., polymerase inhibitors), antifungal agents (e.g., those acting on fungal metabolism or cell wall), biological effectors (e.g., steroids, hormones, growth factors, cytokines, chemokines, etc.), currently approved small molecule drugs, etc. Furthermore, contemplated other components may also be added to modify one or more physical and/or chemical properties. For example, preservatives, wetting agents, detergents, charged groups, etc. may be includes. Among such other components, those are especially preferred that can be in direct contact with a wound (e.g., alginates or other biocompatible polymers, which may or may not be degradable and/or resorbable). Furthermore, and where desired, it should be recognized that various filler materials may be included to achieve a predetermined concentration of contemplated non-porous carbon in the overall composition. Such fillers are preferably inert and can be sterilized. For example, appropriate fillers include cotton fibers, synthetic polymer fibers, clays, silicates, etc.)

Contemplated compositions may be directly applied to a person (e.g., topically applied in form of a powder, or orally in form of a compressed powder or in a capsule), or applied in using a carrier. For example, where the composition is topically applied in a carrier, it is especially preferred that the carrier is in form of a wound dressing in which the non-porous carbon is at least partially enclosed. Therefore, suitable wound dressings include those in which at least one layer of material is between the wound surface and contemplated compositions. Depending on the particular use, the layer may be formed from numerous materials, and particularly suitable materials include gauze (typically cotton), an alginate, and/or a synthetic polymer (e.g., viscose, polyester, etc.).

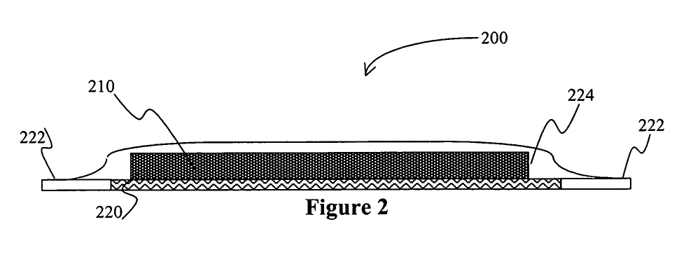

An exemplary wound dressing is depicted in FIG. 2 in which wound dressing 200 has a wound contact layer 220 (e.g., Dermasafe: 66% polyeser, 34% viscose) that together with cotton cover 224 encloses the presently contemplated composition 210. Where desirable, the wound dressing can be prepared in form of a band aid and will then include adhesive elements 222. Of course, it should be recognized that the particular configuration of the wound dressing may vary considerably, and that the ultimate configuration will at least in part depend on the particular use. For example, where the wound dressing covers a relatively small cut or abrasion, adhesive tape may be included. On the other hand, and especially where the wound dressing is held in place by bandaging, no adhesive may be included. Furthermore, the number of layers and materials may change depending on the specific use. For example, dressings for wounds with relatively high degree of exudates may include more than one wound contact layer to provide additional (preferably non-swelling) liquid absorption. On the other hand, dressings for purulent wounds may be relatively thin, and dressings for open wounds may be configured to release at least part of the contemplated composition. It should further be appreciated that while additional layers may be provided to ensure a relatively humid environment for the wound, the many of the compositions according to the inventive subject matter are entirely hydrophobic and may act as a moisture barrier by itself. The term “entirely hydrophobic” composition as used herein means that the composition is water repellent and, when slurried with water and placed on a Buchner filter with tap vacuum for 5 minutes, will retain water in an amount of less than 1 wt %.

In further alternative aspects, the carrier may be provided in numerous forms other than a wound dressing so long as at least some components of the wound will be able to penetrate to the non-porous carbon via one or more of the carrier walls. Where contemplated compositions are orally administered, it should be recognized that all known orally administrable forms are deemed suitable herein. For example, administration may be together with a carrier, disintegrant, lubricant, or other pharmaceutically acceptable additive. On the other hand, administration may also be in form of a crude powder, compacted powder, or slurry (e.g., in fruit juice). Where desirable, contemplated composition may also be incorporated into a capsule, dragee, time release formulation, etc.

Consequently, the inventors also contemplate a method of treating a medical condition in a subject in need thereof, in which contemplated compositions are administered to the subject (e.g., human, farm animal, or pet) in an amount effective to improve at least one symptom of the condition. Where the medical condition is an injury (e.g., open wound, burn, which may be purulent, infected, and/or exuding), symptomatic improvement includes reduced swelling, reduced pain, reduced count of infectious agents, accelerated granulation, and accelerated wound closure. Similarly, where the condition is an allergic condition (e.g., due to bee sting, poison oak, or contact allergy), symptomatic improvement includes reduced swelling, reduced pain, and reduced inflammatory reaction.

On the other hand, where the condition is an intoxication (e.g., alcohol, drug of abuse overdose, chemical compound, and particularly hydrocarbonaceous chemical), diarrhea, and/or, food poisoning, symptomatic improvement includes regaining of consciousness and/or metal clarity, reduction in CFU count in intestinal tract, and/or normalization of stooling. Similarly, and especially where the condition is renal or hepatic dysfunction, hyperuricemia, lactic acidosis, and/or hypercreatininemia, symptomatic improvement includes normalization of hematological parameters, and especially a reduction in serum uric acid and lactic acid levels. In such conditions, the compounds according to the inventive subject matter are preferably administered in a hemodialysis system as well known in the art in which the activated charcoal is replaced with contemplated compositions.

Therefore, the inventors contemplate that contemplated compositions will be associated with an information (e.g., packaging insert, advertising information, or otherwise printed and/or displayed information) that informs a person to apply the composition to an optionally infected open wound, an optionally purulent open wound, an optionally infected burned area, an optionally purulent burned area, and an area with an allergic reaction, or to administer the composition to a person suffering from intoxication, diarrhea, and food poisoning. Alternatively, the information may also announce the fact that contemplated compositions reduce the concentration of uric acid and/or creatinin in the blood or serum.

Further aspects, compositions, methods, and uses are disclosed in our commonly owned copending U.S. applications with the title “Compositions and Methods for Gas and liquid Purification” (filed Dec. 7, 2004) and “Mass Production Of Carbon Nanostructures” (filed Dec. 7, 2004), both of which are incorporated by reference herein.

### EXAMPLES

The following examples are provided only to illustrate selected aspects of the inventive subject matter and are not limiting to the inventive concept presented herein.

### Production of Contemplated Compositions

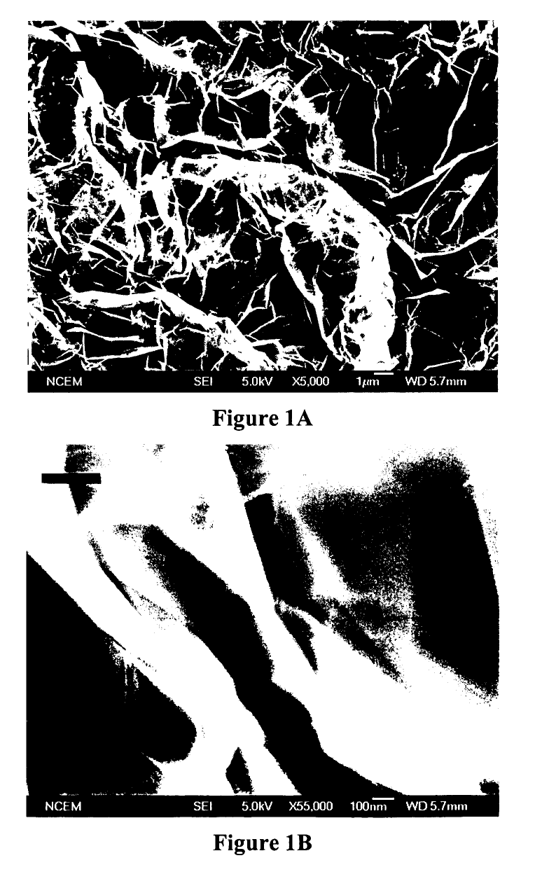

100 g of flake graphite (e.g., commercially available from Superior Graphite Company, 10 South Riverside Plaza, Chicago, Ill. 60606, or Crystal Graphite Corp., Vancouver, B.C., Canada) was admixed with 100 ml activated acid catalyst (e.g., Activated Acid Catalyst #3, commercially available from SupraCarbonic, 348 N. Eckhoff Street—Orange, Calif. 92868, USA) and briefly heated to expansion at about 100° C. to about 200° C. The so obtained material was used without further purification for electron microscopy and exemplary electron micrographs at different magnifications are shown in FIG. 1A and FIG. 1B. Depending on the purity and quality of the graphite, the so obtained material typically comprises between 30 wt % and 99 wt % graphene. Here, the graphene seen as ultra-thin and opaque layer is substantially contorted, while the areas where the sheet is folded and where the fold faces the observer is seen as white reflective lines/areas.

Numerous alternative activated acid catalysts may also be employed for production of contemplated materials. Suitable activated acid catalysts include acidic solutions of a compound, wherein the solution (typically, but not necessarily aqueous) is subjected to an electromagnetic field, electromagnetic radiation, and/or laser irradiation. Most preferably, the activated acid catalyst comprises an acidic solution that is plasma-activated and/or comprises a compound having the general formula MXOₙ, wherein M is selected from the group consisting of H, NH₄, Na, and K, wherein X is selected from the group consisting of Cl, Br, and I, and wherein n is an integer between 0 and 4, inclusive. It should be noted that the term “activated acid catalyst” also includes one or more oxidizers (typically, but not necessarily in aqueous solution) that were previously subjected to electromagnetic radiation, an electromagnetic field, and/or laser irradiation. Alternatively, and in less preferred aspects, activation using the electromagnetic radiation, electromagnetic field, and/or laser irradiation may replaced by admixing the acid or oxidizer with a penetration enhancer (e.g., compounds and/or mixtures commonly found in lubricating formulations, etc.).

The inventors thus discovered that a reagent for carbon-carbon bond cleavage reactions can also be employed to form from various starting materials (e.g., coal, tar, graphite, etc.) a non-porous nanostructured carbonaceous material with a smallest dimension of less than 500 nm, more typically of less than 300 nm, even more typically of less than 200 nm, and most typically of less than 100 nm. In most preferred aspects, such reagents were used to produce graphene from the appropriate starting material (typically graphite). There are numerous carbon-carbon bond cleavage reagents known in the art, and all of them are considered suitable for use herein. However, particularly preferred reagents include commercially available activated acid catalysts (e g., Catalog Item: Activated Acid Catalyst #3 (plasma-activated hydrochloric acid) by SupraCarbonic, LLC., 348 N. Eckhoff Street—Orange, Calif. 92868, USA). Formation of graphene using such reagents is particularly remarkable as “ . . . planar graphene itself has been presumed not to exist in the free state, being unstable with respect to the formation of curved structures such as soot, fullerenes, and nanotubes . . . ” [quoting Novoselov, K. S. et al. “Electric Field Effect in Atomically Thin Carbon Films”, Science, Vol 306, Issue 5696, 666-669, 22 Oct. 2004].

### Medical Uses of Contemplated Compositions

### Materials and Methods

The material obtained as described above was used after steam autoclaving for further evaluation as described below.

Sterile, white male rats were used for all animal experiments. The assessment of wound healing was by in vivo experiments using standard experimental models of purulent and burn wounds. The rats were divided in four groups (control, burn wound; control, purulent wound) with 30 animals each.

### Cytomorphological and Cytochemical Investigations

Cytomorphological and cytochemical investigations were carried out according to P. M. Pokrovskaya and M. S. Makarov in 1942 (1. Pokrovskaya M. P., Makarov M. S. The cytology of wound exudate as an indicator of the wound healing process.—M.: Medgiz, 1942). Smear-prints were taken at every dressing of the wound on the 3rd, 7th, 10^{th}, and 15th day after prior removal of the pus from the wound surface. The smear-prints were Romanovsky-Gimsa stained using buffer solution at pH 6.5. For glycogen test, smears were stained using the periodic acid Schiff reaction of MacManus with control processing of the smears with amylase. For identification of DNA and RNA,the smears were stained with hallocyanogen by the method of Eiparsson with control processing of the smears by the method of Dempsy.

### Microbiological Investigations

Passive shielding using contemplated compositions: Sterile beef-extract agar was used in the study. The agar was covered with non-porous carbon, and the layer of non-porous carbon was as thick as 0.2-0.3 cm. The agar dishes were kept open for 30 minutes to allow settlement of airborne microorganisms on the surface. Simultaneously, the control samples without non-porous carbon were kept open for the same exposure time. The dishes were incubated at 37 ° C. for the period of 48 hours. The results were assessed according to the total number of colonies grown in the experimental and control samples.

Effect of non-porous carbon on the growth and reproduction of microorganisms on blood agar: One-day old test-cultures of microorganisms (Staphylococcus aureus-209, Escherichia coli, Clostridium perfringens-235) were washed off a culture dish with physiological solution and diluted according to the standard scale of turbidity up to 20 U. The method of standard dilutions was used to make the suspension equal to 2.5 U. One standard infected loop taken from the 2.5 U solution (250 ml), was added to 1 mm³ of physiological solution. After this, the diluted culture was inoculated to the blood agar. One ml of the prepared culture was added into an empty dish, covered with the non-porous carbon (0.2-0.3 mm thick layer), and the beef-extract agar was poured over the non-porous carbon at a temperature of 45° C. The control and experimental samples were placed in the incubator for 24 hours (37° C.). The results were assessed according to the total number of colonies grown in the experimental and control samples.

Evaluation of Microbe Contamination in Purulent Wounds: The quantitative analysis was carried out by the method of E. D. Rotheram.

Characteristics of regenerative processes in bone marrow by results of reticulocitosis in the peripheral blood was carried out according to the well known methods by analysis of blood taken from the caudal vein of the animal.

Preparation of plane muscular-cutaneous wounds: Purulent wounds were created as follows: The round piece of skin of the area of 400 mm² together with cellular tissue was cut out in the interscapular area of the rats under ketamine narcosis after their scalp had been removed. The edges of the wound and underlying muscles were squashed using Koher's clamp. The wounds were contaminated with Staphylococcus aureus and Pseudomonas aeruginosa with about 10⁹ microbes per 1 mm. The strains were taken from the patients with purulent complications. In order to avoid contraction of the wounds and difference in initial sizes of wound surfaces in animals, the edges of wounds were fixated with the duralumin ring of 400 m² area. Then the ring was hermetically sealed with cellophane film. After 48 hours the wound surface presented as a nidus of the acute purulent inflammation (Shin F. E. The treatment of purulent wounds of soft tissues with silicon organic absorbent Aerofil and ultraviolet irradiation/Dissertation for MD.—M., 1995.—P. 132).

Thermal burns were produced by the following method (Paramonov B. A. The super-economical skin plastics in the treatment of severely burnt patients/Dissertation for PhD.—St. Petersburg, 1996.—P. 150): In animals under anaesthesia hair was depilated on the back, then pieces of gauze (several layers) moistened with alcohol, put on the depilated area and burnt. The duration of thermal exposure was from 1 to 2 minutes and resulted in formation of a burn wound with a size of 3×4 cm (12 cm²) with complete damage of the skin and cellular tissue—3-4 grade burn.

### Antimicrobial Activity In Vitro

The influence of the non-porous carbon on the growth was evaluated by inoculation of test cultures on the blood agar as described above. The results below clearly indicate a reduction of microbiological growth.

| | Number of | Number of | Difference of the | |
|---|---|---|---|---|
| | colonies | colonies | number of colonies | |
| | in the control | in the | in the control and | % of |
| No. | group | experiment | experiment | difference |
| 1 | 86 | 80 | 6 | 6.98 |
| 2 | 86 | 69 | 17 | 19.77 |
| 3 | 86 | 73 | 13 | 15.12 |
| 4 | 86 | 76 | 10 | 11.63 |
| 5 | 86 | 78 | 8 | 9.3 |
| 6 | 86 | 77 | 9 | 10.47 |
| 7 | 86 | 68 | 18 | 20.93 |
| 8 | 86 | 66 | 20 | 23.26 |
| 9 | 86 | 74 | 12 | 13.95 |
| 10 | 86 | 77 | 9 | 10.47 |
| M ± σ | 86 ± 0 | 73.8 ± 4.7 | 11.9 ± 4.7 | 14.19 ± 5.48 |
| P | | | <0.05 | |

The differences were even more evident in the experiment using airborne inoculation as can be taken from the table below. The observed difference is most likely a combination of a passive protective effect and binding of microorganisms to the non-porous carbon:

| | | | The difference | |
|---|---|---|---|---|
| | Number of | | in the number | |
| | colonies | Number of | of colonies in | |
| | in the control | colonies in the | the control | |
| No. | group | experiment | and experiment | Difference, % |
| 1 | 40 | 11 | 29 | 72.5 |
| 2 | 40 | 4 | 36 | 90.0 |
| 3 | 40 | 4 | 36 | 90.0 |
| 4 | 40 | 8 | 32 | 80.0 |
| 5 | 40 | 7 | 33 | 82.5 |
| 6 | 40 | 4 | 36 | 90.0 |
| 7 | 40 | 5 | 35 | 87.5 |
| 8 | 40 | 5 | 35 | 87.5 |
| 9 | 40 | 7 | 33 | 82.5 |
| 10 | 40 | 6 | 34 | 85.75 |
| \textcircled{1} | 40 | 10 | 30 | 82.5 |
| 12 | 40 | 7 | 33 | 82.5 |
| 13 | 40 | 7 | 33 | 82.5 |
| 14 | 40 | 5 | 35 | 87.5 |
| M ± σ | 40 ± 0 | 6.43 ± 2.17 | 33.57 ± 2.17 | 84.52 ± 4.83 |
| P | | | <0.05 | |

### Antimicrobial Activity in Vivo

In 48 hours after making the injury and infection, the purulent wound presented as a nidus of the acute purulent inflammation with a microbe count of 10×9 CFU/ml in the wound exudate.

On the 3rd day of treatment the wound surface of rats of the control group was characterized by the microbe contamination, which was much higher than the critical level (10⁸⁻⁹ CFU/ml). At the same time the wound surfaces of the animals treated with the non-porous carbon were contaminated with the microbes at the level, in most cases, not exceeding 10⁷ CFU in 1 ml of the wound exudate.

In the control group of animals by the 5th day of treatment the number of colony forming units (CFU) of microorganisms came down to 10×7 and was statistically different from the previous data (P<0.05). The microbiological examination of the wound surface of the rats in the control group revealed the reduction in the number of microbe bodies down to the critical level (10⁵ CFU/ml, P<0.05).

By the 7th day of treatment in rats treated with antiseptic solutions the reduction of the microbe contamination level of the wounds was not observed, and the number was 10⁷⁻⁸ CFU/ml. The level of microbe contamination in the experimental group of animals reduced, and in most of rats the number of CFU did not exceed 10⁴ CFU/ml.

By the 10th day of treatment there is a tendency to the increase of microbe contamination of wounds in the control group, which is, probably, due to the appearance of the associated microflora. By this time the number of microbe bodies in 1 ml of exudate was 10⁸. At the same time the microbe contamination of wounds remained at the same level (10⁴) and was statistically different from those in the control group (P<0.05).

By the 15th day of treatment the level of microbe contamination of wounds in the control group of animals increased up to the critical level and was 10⁵ CFU/ml in most animals. In the experimental group the microbe contamination of wounds was lower than critical level (P<0.05) and did not exceed 10³ CFU in 1 ml in all animals. The data are summarized in the table below:

| | Number of days of treatment and level of microbe | | | | |
|---|---|---|---|---|---|
| | contamination of purulent wounds in rats | | | | |
| | depending on the applied method of treatment | | | | |
| Group of animals | 48 h | 3 | 5 | 10 | 15 |
| Control group | 10⁹ | 10⁹ | 10⁷ | 10⁸ | 10⁵ |
| Experimental group | — | 10⁷ | 10⁵ | 10⁴ | 10³ |

### Treatment of Purulent and Infected Wounds

The experimental full-layer muscular-cutaneous purulent wounds after 48 hours of their appearance and infection with the bacterial suspension of St. aureus and Ps. Aeruginosa presented as nidi of acute purulent inflammation. On primary examination the edges of wounds were bolster-like thickened and undermined. The wound surface was crater-like deepened, while the bottom was covered with friable, easily removable scab. The discharge was abundant, purulent and with a strong ichorous smell. The fascia and muscular fibers underlying the scab were necrotized.

On the 3rd day of treatment the purulent wounds in the control group were characterized by the signs of increasing inflammation. The swelling and hyperemia of adjacent tissues increased. The wound surface was covered with a thick tight-fitting dark-brown scab. After the scab had been removed, the bottom of the wound, filled with purulent fibrous masses, bared itself.

At the same time, the degree of tissue swelling and hyperemia in rats treated with the absorbent non-porous carbon remained at the same level. The wound scab was thick, but not as dense as in the control group. In most animals there was a tendency to changing the character of exudates from purulent to serous-fibrinous.

After 4 dressings (5th day of the treatment) the signs of the acute inflammation in the control group of animals continued to grow. The tissue swelling and hyperemia also increased compared to previous days. The scab was dense and hardly separable from underlying tissues baring the wound bottom, covered with necrotized tissues.

In rats, treated with non-porous carbon, the tissue swelling and hyperemia markedly decreased. The wound defect was covered with a dense thin brown scab. In most animals the scab was easily removable from underlying tissues. After the scab had been removed, the bared wound surface showed no signs of the presence of purulent masses, while solitary dark-pink nidi of granulation tissue were found. The exudate was scarce and serous-fibrous.

After 7 days of treatment the signs of the acute purulent inflammation in the control group of animals still persist. The edges of the wound are thickened, immovable and undermined in many animals. The scab is dense and dark-green. After its removal there are large masses of necrotized tissues. The amount of exudates is slightly lower compared to previous days of the experiment, but it still has purulent character and ichorous smell.

At the same time in rats having been treated with the non-porous carbon there was only slight tissue swelling and hyperemia. In all animals the wounds were covered with the scab easily removable from underlying tissues. Under the scab there were dark-pink nidi of granulation tissue. The exudate was scarce and serous-fibrous.

By the 10th day of treatment there was a decrease in swelling and inflammation of tissues around the wound in the control group of animals. The process of separation of the scab from the underlying tissues started with the bared wound filled with necrotized tissues. However, there were some pale-pink areas with granulation tissue as well. The exudates is still purulent, but its amount is scarce and mostly in the periphery of the wound and in its side pockets. The area of the wound decreased to 29.03±13.7 mm² due to the contraction of the wound edges. The area was 73% compared to its initial size.

At the same time the absence of tissue swelling and hyperemia around wounds was characteristic in rats treated with the non-porous carbon. The thin light-brown scab is mostly easily removable from the underlying tissues, with the bared wound covered with granulation tissue. There is well a marked border of edge epithelization in the periphery of the wound. The skin around the wound has a star-shaped folding and is easily movable. By this day of treatment the surface area of the wound in the experimental group was 198.6±8.1 mm² (48.0% compared to initial). The appearance of the border of edge epithelization was considered as the indication to stop the local absorbing treatment of purulent wounds. The consequent treatment was continued using ointment dressings.

By the 15th day of treatment all the signs of inflammation disappeared in most animals in the control group (except 6 rats). The wound surface was much less in size compared to previous days of the experiment and was 187.3±10.2 mm² (46.3%). In animals treated with the absorbent the wound surface decreased to 95.1±4.4 mm² (20.4%), which was statistically different from the indicant in the control group (P<0.01). The border of edge epithelization was markedly visible in all animals.

In 20 days the wound was completely filled with granulation tissue in the control group, the exudate was scarce and serous. There was an active epithelization process in the edges of the wound. The area of the wounds was 94.5±4.2 mm² (23.3%). The wound healing process in the carbon treated group of animals in the same period of time was characterized by further reduction of the wound surface area and was 9.2±0.3 mm² (2.3%), which was statistically different from the indicants on the control group (P<0.05). The final complete healing of the wounds in the control group was on 28.3±0.5 day, while in the experimental group—23.2±0.3 day, with the acceleration of healing process of 34.3%.

Thus, it should be recognized that treatment with non-porous carbon of purulent wounds reduces the tissue swelling, which results in earlier cleaning of the wound surface from purulent necrotic masses, and creates favorable conditions for the development of granulation tissue and edge epithelization, which occurs significantly earlier than in animals not treated with the non-porous carbon.

### Morphological Tests in the Control Group

In order to investigate the changes of structure of the wound tissues we carried out morphological investigations of the biopsy material taken from the wound surfaces of the animals of the control and experimental groups. The control group had animals with purulent wounds, treated with solutions of antiseptics and ointment dressings.

3-5 day. The wound surface is filled with scab, which covers the tissue detritus and fibrinous exudates, infiltrated with large amount of destructed leukocytes. There are numerous colonies of microorganisms in the leukocyte-fibrinous layer and under it. The agents of wound infection are actively phagocyted by neutrophiles and macrophages. The bottom of the wound consists of the large amount of cellular tissue.

The superficial layers of cellular tissue are destructed, which is proven by the presence of numerous granules of deformed lipocytes. Neutrophiles are the prevailing cells both in superficial and deeper layers of tissues. Only in small part of neutrophiles glycogen granules are revealed using periodic acid Schiff reaction, which means that the processes of intracellular metabolism are affected. At least ⅓ of all leukocytes are in the state of disintegration. The muscular fibers adjacent to cellular tissue are also destructed, swollen and infiltrated with a lot of leukocytes.

Small part of macrophages is represented both by small immature forms and bigger cells. Cytoplasm of the latter, as revealed using periodic acid Schiff reaction, is rich in vacuoles and additional elements, which proves their functional activity. There is a tendency towards increase in the number of phagocyting macrophages by the 5th day of treatment. Mast cells are mainly present around blood vessels and are characterized by the presence of a compact metachromatic granular structure, which is revealed after staining them with blue toluidine. It should be mentioned that most part of mast cells have orthochromatic granular structure and cytoplasm with the signs of degranulation and vacuolization, which signifies about their disintegration. In this period the proliferation of fibroblasts and formation of new capillaries through budding were revealed in some areas of cellular tissue, mostly in the edges of the wound. However, the vascular elements are not characterized by a vertical tendency. There are fibroblasts growing disorderly in the deeper layers of the wound and in the cellular tissue, near which immature collagen fibers are found. They are argyrophil after argentation and metachromatic after staining with blue toluidine. There is also revealed the metachromatic main substance, which means that fibroblasts synthesize acid aminoglycans. The signs of hemorrhages, hemostasis, increased vascular permeability, microthrombosis, sludge-syndrome and diapedetic hemorrhages are also revealed in the blood vessels of the newly formed tissue.

7-10 day. By this time the wound defect is already filled with granulation tissue with typical vertical blood vessels. In comparison with the previous term of the study the degree of maturity of the tissue is getting higher through this period. There are four distinct layers in the granulation tissue: leukocyte-fibrinous layer, layer of vascular arcades, layer of lower vessels, horizontal fibroblasts and layer of fibers.

Neutrophiles are the prevailing cells in the superficial layers. The number of macrophages increased compared to the 5th day, with the most of them represented by large forms in the state of active phagocytosis. They are mostly found in the layer of vertical vessels.

In the layer of horizontal fibroblasts the latter are located parallel to the wound surface. After performing Brashe reaction the pironinophily of cytoplasm and nucleoli is marked, meaning that fibroblasts synthesize RNA and protein products (collagen, etc.). The metachromatic character of the main substance is due to the accumulation of acid glycoseaminoglycans. The tinctorial properties of growing collagen fibers are also changed, which is manifested by their reduced metachromasy and argyrophily and the presence of fuchsinophily. These processes reach their maximum activity by the 10th day. In the layer of vertical vessels the fibroblasts have no distinct orientation, while the main substance is characterized by the slight metachromasy. The signs of microcirculatory disturbance still persist: vascular dilation, signs of hemo- and lymphostasis, disturbance of vascular permeability, sludging of erythrocytes.

As in the previous terms of the study there are signs of plasmorrhages and microhemorrhages. There are microabscesses and secondary granulation tissue necroses in the different parts of the tissue. The numerous colonies of microorganisms surrounded by lots of destructed neutrophiles are found in the leukocyte-fibrinous layer. However, this layer is getting thinner. This means that the acute inflammatory reaction still persists. There are congregations of netrophiles even in the forming layer of horizontal fibroblasts. The fibrosis of these areas is inhibited, which means that the process of maturing of collagen fibers is retarded. The areas with wrongly oriented fascicles of fibroblasts and collagen fibers are often found. Besides, as in the previous terms of the study, there are numerous abscesses and nidi of the secondary tissue necrosis. By the 10th day of the treatment the number of the colonies of microorganisms is less. The microcirculatory disorders are regressing, which is manifested by the reduction of microthromboses and signs of sludge-syndrome. However, there is still marked tissue swelling and some microhemorrhages in the superficial layers and the layer of vertical vessels, which are due to the increased vascular permeability. There are congregations of lymphocytes and plasmatic cells in the vicinity. The edges of the wounds are characterized by the picture of active regeneration of damaged epidermis. The spare edge of the latter covers the granulation tissue to some extent and partly crawls over the fibrinous exudate. The epithelial cells contain acid Schiff-positive granules of glycogen. In the whole the epidermis is characterized by a steady regeneration of the vertical anisomorphism.

20th day. The process of maturing of the granulation tissue and its epithelization goes rather sluggishly alongside with the formed layer of vertical vessels and numerous neutrophiles. At the same time the phenomena of fibrosis of horizontal fibroblasts are progressing alongside with the increasing number of the mature fuchsinophile collagen fibers. Besides, there is an increase in the number of low-active fibroblasts with unmarked pironinophily of cytoplasm. At the same time there is a friable layer of large fibroblasts in the layer of vertical vessels. The main substance is markedly metachromatic. The growing of collagen fibers in this layer is retarded. There are some microabscesses and nidi of the secondary necrosis in the granulation tissue. The regenerating epidermis crawls over the granulation tissue in the edges of the wound. However, most of the granulation tissue is still not epithelized.

Only by the 30th day since the beginning of the treatment the epithelization of the wound occurs. The scar tissue is under the newly formed epidermis and contains not numerous blood vessels and fibroblasts, most of which are not active fibrocytes. In most animals the epithelization is not complete. There is a wound surface in the center covered with the secondary scab and a thin layer of fibrin. There is a granulation-fibrose tissue under it with the signs of the local neutrophile, macrophagal, lymphocyte and plasmatic cell infiltration. All this indicates about the prolongation of the inflammatory processes and inhibition of the reparative processes in animals with the experimental model of the purulent wound.

### Morphological Tests in the Group Treated with Non-Porous Carbon

3-5 days. The using of the non-porous carbon in the treatment of purulent wounds in rats in the early terms speeds up the regeneration process. The removal of the tissue detritus from the surface of the wound occurs earlier than in the control group of animals, which in its turn reduces bacterial contamination and inflammation and increases the regeneration processes. This is manifested by the significant decrease of the signs of microcirculatory disorders, which is found in the biological specimens taken from the wound surface of the animals treated with the non-porous carbon, after 3-5 days following the injury and infecting. There is less marked infiltration of the cellular tissue and newly formed tissue in these terms of the treatment. However, alongside with the intensification of the processes of fibroblast proliferation and angiogenesis the signs of lympho- and hemostasis are revealed less frequently. The granulation tissue of full value is formed by the 5th day, with the layer of horizontal fibroblasts and vertical vessels found inside the tissue. Moreover the fibroblasts are characterized by a high content of RNA and relatively high fibrillogenesis. The granulation tissue replaces vast areas of the cellular tissue. There is an intensification of the macrophagal and mast cell response.

The wound surface is covered with a relatively thin leukocyte-fibrinous layer. In the latter, compared to the one in the control group, the colonies of microorganisms, microabscesses, hemorrhages and areas of the secondary tissue necrosis are found much less frequently.

7-10 days. The maturing of the granulation tissue is progressing. By the 7th day it occupies the whole area of the wound defect, replacing the cellular tissue. Yet in this period all the layers of the tissue are well formed. The narrow fibrinous-leukocyte layer is practically devoid of the colonies of microorganisms and necrotic masses. The number of neutrophiles is decreased, especially destructed ones. There are signs of fibrosis revealed in the deeper layers of the granulation tissue, which intensifies by the 10th day, while in the layer of horizontal fibroblasts there are fascicles of the mature collagen fibers.

The main substance is less metachromatic in these areas. The cytoplasm and nucleoli of fibroblasts are characterized by the reduction of pironinophily, which means that the nucleoli are transformed into the low-active fibrocytes. There are polymorphic nuclear leukocytes in the tissue. However, the number of these cells is less than in the control group. There are rather numerous neutrophile leukocytes in the more superficial areas, which is the layer of the vertical vessels. However, there are also functionally active fibroblasts in this area. In comparison to the previous group of animals this group is characterized by the increase in the number of macrophages with acid Schiff-positive foamy cytoplasm. The disorders of microcirculation are less severe than in the previous group, as well as the number of microabscesses and secondary tissue necroses is reduced. The colonies of microorganisms are extremely rare. The regeneration of the epidermis, visible since the 7th day, is more active compared to the control group.

15th day. The fibrosis and epithelization of the granulation tissue are progressing. In most animals the layer of the vertical vessels is almost completely replaced by the fibrosed layer of horizontal fibroblasts, i.e. the fibrinous-scar transformation occurs. The functionally active fibroblasts with pironinophile cytoplasm and nucleoli are found only in the very superficial areas of the wound. Most of all the fibroblasts are transformed into functionally low-active fibrocytes. There are thick, in some parts twisted, mature collagen fibers, stained in red color by the method of Van-Gisone.

### Smear Print Analysis of Animals Treated with Non-Porous Carbon

In 48 hours after the injury and infecting the wound was characterized by the development of the acute associated microflora with staphylococci prevailing. The latter were located both diffusely and forming separate conglomeration between the fibrin fibres and destructed leukocytes. The attention was drawn by the presence of the large number of neutrophiles in the wound exudate, which were in the state of incomplete and degenerative phagocytosis (up to 32.7% in most examined smear-prints). From 10 to 50 staphilococci were revealed in their cytoplasm and nucleoli. Only in some neutrophiles from 8 to 25 microbe bodies of the Gram-negative microflora were detected. The presence of the large number (40-70) of staphylococci in the sites of leukocytes destruction was the evidence of the degenerative type of inflammatory reaction. The active migration of neutrophiles to the wound exudate was characteristic. The number of such cell elements in all the view fields was at least 35.4±1.2%, with the most of them being in the state of necrosis and dystrophic changes (67.9±1.5% and 27.0±3.1% accordingly). The number of relatively intact neutrophiles was small (in average not more than 8.2±0.7%). The small number of mononuclear cells was also the evidence of the acute purulent inflammation. In all the smear-prints of the wound studied the number of polyblasts and macrophages did not exceed 2.5±0.2%.

3rd day of treatment. The cytological investigation of the smear-prints from the wounds of the rats treated with the non-porous carbon showed the reduction in the number of microorganisms in the wound exudate. Up to 30 staphilococci and up to 16 Gram-negative bacilli had been detected in neutrophile leukocytes. The number of leukocytes in the state of incomplete phagocytosis and degenerative changes was much less (8.3±1.3%), as well as destructed neutrophiles (50.0±2.1%). At the same time the number of intact neutrophiles increased is (28.5±9.4%) and mononuclear elements (27.9±1.0%) increased. The decrease of the migration of leukocytes to the wound exudate in the rats, treated with the non-porous carbon was the evidence of the reduction of the inflammatory process. This index was statistically lower in, practically, all the smear-prints, compared to the previous days (16.8±0.9%, P<0.05).

At the same time the number of microorganisms in the wound exudate of the control group had significantly grown up. The microorganisms formed the numerous conglomerates in the intercellular space. The conglomerates were separate colonies with dense centers from which the chains of microbes' bodies stretched out like rays. It is worth to mention that during this term of the study the change of the microbiological landscape was revealed in rats treated with conventional methods. The number of bacilli-like microorganisms detected inside the neutrophile leukocytes (60-80 microbe bodies) exceeded the number of staphylococci (30-45 microbe bodies). The number of neutrophiles in the state of incomplete phagocytosis and degenerative disintegration reduced almost twice. But their number still exceeded significantly the one in the experimental group of animals (15.9±2.3%). Besides, some increase of the percentage of leukocytes migrating to the exudate witnessed of still persisting acute purulent inflammation (42.3±1.2%). The investigations of the smear-prints revealed, that during this term of treatment in the control group the increase in number of intact neutrophiles and proliferative cells of the connective tissue is not observed (8.7±2.8% and 2.9±0.4% accordingly).

5th day of treatment. The number of free and intracellular microorganisms in the smear-prints of the experimental group of animals reduced to solitary microbe bodies in some view fields. Only in some smear-prints the neutrophile leukocytes contained up to 10-12 Gram-positive bacilli and 4-6 staphilococci. Compared to the previous days of the study the significant reduction of the number of neutrophiles in the state of incomplete phagocytosis (3.8±1.7%) was observed.

During this term of the study the number of neutrophiles migrating to the wound exudate was 13.2±2.2% (P<0.05). The increase of the percentage of the normally segmented neutrophiles for up to 68.1±4.7% and mononuclear cells for up to 31.5±0.8% (P<0.05) was evidenced for the regression of the inflammation process. It is worth to mention that in the early terms of the treatment out of all the connective tissue cells of reparation the number of polyblasts and profibroblasts was not less than 26.1±0.9%.

At the same time the number of both extra- and intracellular located microorganisms was significantly higher in the control group of rats compared to the experimental group (30-40 and 15-20 accordingly in the most of studied smear-prints). Despite of the 5th day of the treatment the numbers of intact neutrophiles (2.4±3.3%) and mononuclear cells (10.1±0.8%, including 7.2±1.3% of polyblasts and profibroblasts) were still low.

10th day of treatment. By this time there were still some microorganisms found in the smear prints of the rats from experimental group. However, their quantity as well as the quality state correlated well with the general clinical picture of active epithelization of the wound surface. Only in 3 smear-prints several neutrophiles were found, which contained from 4 to 10 staphilococci bodies in their cytoplasm and nucleoli. The number of neutrophiles in the state of incomplete phagocytosis and degenerative changes was not less than 1.9±0.3%. The numbers of neutrophiles (12.6±0.6%) and mononuclear cells (77.4±2.7%) migrating to the wound exudate proved the favorable effect of the non-porous carbon on the wound healing process.

There were no significant differences revealed on cytological examination of the wound exudate in animals from the control group compared to the previous days. In most cases both free and intracellular microorganisms (not less than 10-25 microbe bodies) as well as fibrin threads and destructed neutrophiles were detected in the smear-prints. The number of neutrophiles in the state of incomplete phagocytosis and degenerative changes was 4.2±1.1%, while the number of those migrating to the exudate was 36.9±2.3%. These facts as well as the small number of segmented neutrophiles (9.6±3.3%) and mononuclear cells (8.3±2.1%) evidenced about slowing down of the wound healing process.

Thus, the cytological examination of smear-prints of the wounds of rats treated with the non-porous carbon clearly indicated favorable effects on the dynamics of the acute inflammation process (reduction of severity and period of inflammation).

### Treatment of Purulent and Infected Burns

The local treatment of wounds was started immediately after making the thermal injury. The dressings were made daily until the complete clearing of the wounds. After the end of necrolisis and complete healing the dressings were made every second day. The course of the wound healing process was assessed by the results of the visual observation, calculating the degree of the acceleration of the wound clearing and wound healing compared to the control group. The visual assessment of the course of wound healing process is based on the dynamic observation for the changes in the wound, which allows to determine the terms of clearing, activity of the inflammation, degree and character of exudation, the speed of the decrease of wound defect and the terms of the complete healing. The clinical study of the processes of reparation helps to draw a full picture of its course. However, in order to detect more delicate changes in the tissues caused by a number of factors, which cannot be differentiated visually one needs to use special methods. According to Russian and foreign publications we can conclude that studying the histogenesis of wounds is sufficient for this kind of works.

In order to determine the area of burn we used the formula suggested by M. Lee (1929): S=12.54×WO.66; where S—surface of the body of the rat, cm² and W—weight of the animal, g. The comparative assessment of the wound healing process was carried out according to the degree of necrolisis and wound healing using above-mentioned treatment. This index was calculated by the following formula: T/T1×100% where T is duration of necrolisis (healing) in the experimental group, and T1 is duration of necrolisis (healing) in the control group

The histological and histochemical tests were carried out after decapitation of the animals in the following terms: 3, 5, 5, 10, 15 and 21st day of treatment. The samples of tissues with the size of 0.5×0.5 cm were taken from the wound surface. The tissue samples were fixated in the Carnoi's liquid, after that they were poured in paraffin. Then sections were made. The sections were stained with hematoxilin-eosine, pichrofuchsine by Van-Gisone, impregnated with silver by Gomeri. The histochemical investigations were based on the detection of glycoseaminoglycans (staining with toluidine blue), neutral muchopolysaccharides and glycogen (acid-Schiff reaction), RNA (staining with pironine green by Brashe). Results are summarized in the table below:

| | Results of treatment | | | |
|---|---|---|---|---|
| | Necrolisis | | Healing | |
| | Average | Acceleration | Average | Acceleration |
| Group of | terms, | compared to | terms, | compared to |
| animals | days | the control, % | days | the control, % |
| Control group | 12.5 ± 1.6 | — | 29.6 ± 1.3 | — |
| Experimental | 9.4 ± 1.8 | 24.8 | 26.2 ± 1.5 | 11.5 |
| group | | | | |

As can be clearly seen, the non-porous carbon treatment of burn wounds shortened the terms of treatment for 3-4 days compared to the control group of animals. It is worth to mention, that there was no significant difference in the character of clinical changes in burn wounds in rats compared to purulent wounds in these animals. The only difference was some delay of the complete clearing and healing of the wounds.

Mortality: The mortality rate in the control group was 30%. In most cases, the death of the animals occurred from 7th to 30th day after the burn. All the dead rats were characterized by the extreme degree of exhaustion and had deep burn wounds, complicated with the purulent infection. At the same time covering of the burn wounds with the absorbing carbon material provided a significant reduction of the mortality (up to 10%). It is worth to mention, that death of rats was observed during 5 days following the beginning of the treatment. It was, probably, due to the development of the burn shock in those animals.

Hemodynamic changes: A statistically significant difference in wound healing was observed during the course of post-traumatic anemia, and the results are presented in the table below:

| | Groups of animals and duration of the investigation | | | | | |
|---|---|---|---|---|---|---|
| | Control group | | | Experimental group | | |
| Investigated indices | 2^{nd} week | 3^{rd} week | 4^{th} week | 2^{nd} week | 3^{rd} week | 4^{th} week |
| Erythrocytes | 82 ± 5.0 | 74 ± 3.5 | 71 ± 3.0 | 92 ± 1.0 | 86 ± 2.0 | 95 ± 3.0 |
| Hemoglobin | 81 ± 3.0 | 87 ± 2.0 | 89 ± 1.0 | 91 ± 2.0 | 92 ± 1.0 | 96 ± 1.0 |
| Reticulocytes | 77 ± 3.0 | 123 ± 1.0 | 120 ± 1.0 | 124 ± 2.0 | 101 ± 2.0 | 121 ± 3.0 |
| Leukocytes | 150 ± 2.0 | 110 ± 2.0 | 116 ± 2.0 | 123 ± 3.0 | 107 ± 2.0 | 104 ± 1.0 |

As can be clearly seen, in rats treated with antiseptics and ointment applications was a significant reduction in the number of reticulocytes by the end of the second week of observation. As far as this reduction was accompanied by the progressing anemia (reduction in the number of erythrocytes by 20% by 28-30th day), one can assume a depression of the regenerative processes of hemopoesis in bone-marrow. In contrast, reticulocytosis is observed after closing the of burn wounds in the group treated with the non-porous carbon, which is probably due to the absoring properties of lactic acid and other toxic or inhibitory compounds on the wound surface. Also, leukocytosis was more significant and sustained longer at high level in the control group of rats. The application of the non-porous carbon provided normalization of leukocytosis much earlier than in the control group. Thus, it should be recognized that application of the non-porous carbon to burn wounds provides a significant reduction of mortality in animals and shortens the duration of treatment.

### Biopsy Analysis from Burn Wounds

Animals in the control group received conventional treatment for burn wounds. 3rd-5th days of treatment. The burn wound is covered with the homogenous oxophile necrotic scab spreading to the papillar and reticular layers of derma. The surrounding tissues of derma and cellular tissue are swollen and plethoric. The solitary and merging hemorrhages are found. The necrotized tissues are separated from the intact areas by the demarcation leukocyte border. The islets of the forming granulation tissue are found in the deep layers of derma and cellular tissue. The latter is presented by disorderly located capillaries and a lot of cell elements with neutrophiles prevailing. The macrophages, involved in the absorbing of lipid granules of damaged lipocytes are found much less often.

By the 5th day of treatment the fragments of the burnt oxiphile homogenous scab, located among fibrin and numerous neutrophiles, are found on the wound surface. The congested blood vessels, swelling, islets of the forming granulation tissue with a lot of cell elements and newly formed vessels are found in the adjacent areas of derma and cellular tissue. Neutrophiles and macrophages, involved in the absorbing of lipid granules of damaged lipocytes are prevailing.

Fibroblasts are found much less often. In the periphery of the wound there are solitary vertical vessels with acid-Schiff positive fibroblasts located between their loops. After staining with toluidine blue the local metachromasy of the granulation tissue is revealed, which means the content of glycoseaminiglycans is high. The signs of leukostasis, plasmatic impregnation of walls and migration of leukocytes are visible in the vessels of the forming granulation tissue.

In 10 days after the beginning of treatment the fibrinous-leukocyte layer with some fragments of scab is revealed on the wound surface. Under this layer the demarcation leukocyte border is found, which separates the superficial layers of the wound from the typical granulation tissue formed within derma and cellular tissue.

By the 15th day the wound surface is presented by the fibrinous-leukocyte scab with the flat epithelium crawling under it together with the edge of the wound. The granulation tissue is formed within the derma and cellular tissue. It contains a lot of newly formed capillaries, macrophages and fibroblasts, as well as the numerous neutrophiles and lymphoid cells.

By the 27th day the regeneration of the epithelium occurs, which is presented by the flat multi-layer epithelium with a superficial desquamation. The maturing of the granulation tissue goes very slowly. Although there are some fuchsinophile collagen fascicles in deeper layers, the layer of the vertical vessels with some neutrophiles among them is still present.

Thus, the wound healing process in experimental burns in rats lasts for 4 weeks and is characterized by the steady reduction of inflammation, normalization of microcirculation and clearing of the wound from devitalized tissues. At the same time the islets of the granulation tissue appear filling the defect. The proliferation is stimulated and the fibrosis of the most mature areas of the granulation tissue occurs. By the end of the 4th week the epithelization of the wound is completed.

The morphological picture of tissue samples taken from the animals treated with non-porous carbon was similar to that from the control group. However, by the 5th day of treatment the wound surface was characterized by the presence of homogenous oxiphile scab spreading all over derma. At the same time the areas adjacent to the wound are still swollen and plethoric. The demarcation leukocyte border separating the area of the necrosis from cellular tissue was less marked than in the control group. There are stases, perivascular diapedeses and local hemorrhages.

At the same time there are islets of the forming granulation tissue found in cellular tissue and derma. It consists of a lot newly formed capillaries and cell elements, among which neutrophiles, lymphoid cells and macrophages prevail. It is worth to mention, that odd macrophages and fibroblasts, located among leukocytes, are in the state of proliferation, which means the reparation processes are activated, while in the most mature areas of the newly formed tissue the vertical vessels are formed.

In most cases by the 10th day of treatment the wound surface was completely cleared from the burnt scab and was presented by a narrow fibrinous-leukocyte layer with the formed granulation tissue and vertical vessels under it. The number of fibroblasts with acid-Schiff positive cytoplasm, located between layers, increased. The deeper layers of the granulation tissue contain fuchsinophile fascicles of collagen, which indicates the maturing of the granulation tissue. The neutrophiles and remnants of tissue detritus were found on the surface of the granulation tissue. However, there were less of them compared to the previous days.

After 15th day the epithelium starts crawling over to the center of the wound. There are still some fragments of the fibrinous-leukocyte layer in the superficial layers of the granulation tissue. At the same time the number of blood vessels and cell elements in the deeper layers of the granulation tissue decreased. At the same time the number of collagen fibres grows. There are distinctly visible vertical vessels and layer of horizontal fibroblasts in the deeper layers of the wound surface. The number of neutrophiles and macrophages in the layer of vertical vessels decreases, while the number of active fibroblasts increases. The reduction of number of blood vessels in the newly formed tissue is due to their replacement by collagen fibres. The stroma is distinctly metachromatic, which means the content of glycoseaminoglycans is high.

By the 23rd day after the beginning of the treatment the wound surface is completely epithelized in most animals. The granulation tissue is presented by a lot of fuchsinophile fascicles of collagen, forming the scar tissue. The epithelial cells are well differentiated and do not contain granules of glycogen. The solitary, not vertically oriented blood vessels as well as the solitary neutrophiles are found under the epidermis alongside with numerous fibrocytes.

Thus, it was again observed that treatment with non-porous carbon had a significant effect on the wound healing processes in burn wounds (e.g., significant acceleration of the clearing).

### Treatment of Human Plasma

Plasmapheresis was conducted using a rotary pump “Hambro” and slit-shaped nozzles. Peripheral blood from patients was separated by a plasma-separator, PF-05 (Biofizapparatura) according to standrad operating procedures. The so obtained plasma was then passed through a chamber containing contemplated non-porous carbon, and the carbon was removed by skimming and subsequent filtration.

Of 13 analyzed parameters, significant changes were observed for concentrations of uric acid, which decreased more than 50%, lactic acid (data not shown), and for creatinine, which decreased more than 10%. Remarkably, other parameters, including electrolytes, liver enzymes, bilirubin, glucose, and lipid composition were not affected. Therefore, it should be recognized that contemplated compositions may be employed as a treatment modality for hyperuricemia and related conditions (e.g., gout, secondary metabolic changes due to alcoholism, etc.). Furthermore, as renal and hepatic insufficiency tend to increase the level of uric acid and lactic in blood, contemplated compositions may be used for (supplemental) treatment of renal and hepatic insufficiency.

Thus, specific embodiments and applications of compositions and methods for medical use of graphene-containing compositions have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. A medical composition comprising a non-porous carbon other than a fullerene or a nanotube, and in which the carbon has a smallest dimension of less than 100 nanometer.

2. The medical composition of claim 1 in which the carbon has a smallest dimension of less than 20 nanometer.

3. The medical composition of claim 1 in which the non-porous carbon comprises graphene.

4. The medical composition of claim 3 comprising at least 10 wt % graphene.

5. The medical composition of claim 3 comprising at least 50 wt % graphene.

6. The medical composition of claim 1 formulated for topical administration.

7. The medical composition of claim 6 further comprising an information that is associated with the composition, and wherein the information informs a person to apply the composition to an area selected from the group consisting of an optionally infected open wound, an optionally purulent open wound, an optionally infected burned area, an optionally purulent burned area, and an area with an allergic reaction.

8. The medical composition of claim 1 formulated for oral administration.

9. The medical composition of claim 8 further comprising an information that is associated with the composition, and wherein the information informs a person to administer the composition to a person suffering from a condition selected from the group consisting of an intoxication, diarrhea, and food poisoning.

10. The medical composition of claim 1 formulated for contact with blood or serum.

11. The medical composition of claim 10 further comprising an information that is associated with the composition, and wherein the information informs a person that the composition reduces concentration of at least one of uric acid and creatinin in the blood or serum.

12. A wound dressing comprising the composition of claim 1 at least partially enclosed in a carrier, and wherein at least one layer of material is disposed between the wound and the composition of claim 1.

13. The wound dressing of claim 12 wherein the at least one layer comprises a material selected from the group consisting of gauze, an alginate, and a synthetic polymer.

14. A method of treating a medical condition in a subject in need thereof, comprising a step of administering the composition of claim 1 in an amount effective to improve at least one symptom of the condition.

15. The method of claim 14 wherein the condition is selected from the group consisting of an optionally infected open wound, an optionally purulent open wound, an optionally infected burned area, an optionally purulent burned area, and an area with an allergic reaction.

16. The method of claim 15 wherein the composition is topically administered.

17. The method of claim 14 wherein the condition is selected from the group consisting of an intoxication, diarrhea, and food poisoning.

18. The method of claim 17 wherein the composition is orally administered.

19. The method of claim 14 wherein the condition is selected from the group of renal dysfunction, hepatic dysfunction, hyperuricemia, lactic acidosis, and hypercreatininemia.

20. The method of claim 19 wherein the composition is contacted with blood or serum via a membrane that is impermeable for cellular components of blood.
